(19) United States
(12) Patent Application Publication
(10) Pub. No.: US 2023/0099101 A1
(43) Pub. Date: Mar. 30, 2023

(54) SERINE PROTEASE INHIBITORS FOR SUPPRESSING OR PREVENTING ANAPHYLACTIC REACTION

(71) Applicant: The Regents of the University of Michigan, Ann Arbor, MI (US)

(72) Inventors: SIMON P. HOGAN, Ann Arbor, MI (US); AMNAH YAMANI, Ann Arbor, MI (US)

(21) Appl. No.: 17/908,621

(22) PCT Filed: Mar. 5, 2021
(86) PCT No.: PCT/US2021/021018

§ 371 Date: Sep. 1, 2022

Related U.S. Application Data
(60) Provisional application No. 62/986,149, filed on Mar. 6, 2020.

(51) Int. Cl. C12N 15/113; C07K 16/38; A61P 37/08

(52) U.S. Cl. CPC C12N 15/113; C07K 16/38; A61P 37/08; C12N 2310/141; C12N 2310/14; C12N 2310/531; A61K 39/00

(57) ABSTRACT

The present invention relates to novel target pathways for the treatment or prevention of anaphylaxis in a subject. In particular, the invention identifies antagonism of SerpinA3 as a novel target for the treatment of prevention of anaphylaxis in a subject.

### RELATED APPLICATIONS

This application claims priority to and the benefit of U.S. Provisional Patent Application No. 62/986,149 filed Mar. 6, 2020, which is incorporated herein by reference in its entirety for all purposes.

### FIELD OF THE INVENTION

The present invention relates to novel target pathways for the treatment or prevention of anaphylaxis in a subject. In particular, the invention identifies inhibition of SerpinA3 as a novel target for the treatment or prevention of anaphylaxis in a subject.

### BACKGROUND OF THE INVENTION

Anaphylaxis is a severe, life-threatening allergic reaction that affects both children and adults and males and females in the United States. The most common inciting agents (33.2% of reactions) are foods, particularly peanuts and tree nuts, and food-induced anaphylaxis (FIA) hospitalization rates for children in US have more than doubled from 2000 to 2009.

A food-induced anaphylactic reaction encompasses a variety of symptoms that may affect one or more target organs including those of the gastrointestinal (GI), cutaneous, respiratory, and cardiovascular systems. In humans, compromise of either the cardiovascular or respiratory system defines a severe reaction, and it is postulated that basophil- and mast cell (MC)-derived mediators, through inducing pulmonary venous vasodilatation and fluid extravasation, cause the respiratory and cardiovascular collapse that leads to the severe, life-threatening anaphylactic phenotype. Fluid extravasation in anaphylaxis is thought to be consequence of capillary fluid leak due to loss of the vascular endothelial (VE) barrier integrity, leading to the movement of fluids, electrolytes, and proteins from the vascular compartment into the interstitial spaces.

The VE barrier is maintained by adherens junction (AJ) and tight junction (TJ) proteins. The AJ proteins are the most ubiquitously expressed endothelial cell-cell junctional proteins and act as mechanical anchoring points that promote endothelial TJ protein-protein interactions and interjunctional integrity. The TJ proteins are tethered to the actin cytoskeleton and seal the intercellular space, establishing the dense “fence” barrier preventing the bilateral apical-basolateral passage of ions, proteins, and lipids. VE-cadherin is one of the first endothelial cell-specific molecules expressed and required for endothelial survival, blood vessel assembly, and stabilization. VE-cadherin forms Ca²⁺-dependent homophilic interactions with adjacent endothelial cells via actin-linking catenin family proteins and the actin cytoskeleton, establishing the vascular barrier integrity. The stability of the VE-cadherin-catenin-cytoskeleton complex is essential to maintaining endothelial barrier function and disruption of these processes via receptor-signaling pathways including non-receptor kinases, including SRC, ABL1 and ARG and myosin light chain kinase (MLCK) leads to VE-cadherin-mediated AJ disorganization or VE-cadherin internalization and loss of endothelial barrier integrity.

The cellular and molecular pathways that directly contribute to the severe anaphylaxis phenotype are unclear. Clinical studies have reported increased levels of IL-4 and histamine in the serum of human patients with severe anaphylaxis, suggesting a possible role for these molecules in expression of the severe disease phenotype. However, the cellular target of these IL-4-mediated effects and the underlying signaling processes involved in the amplification of VE barrier dysfunction and fluid extravasation is not yet fully understood. Accordingly, there remains a need for identification of novel cellular mechanisms that cause or contribute to anaphylaxis, and therapeutic agents to target the same.

### SUMMARY OF THE INVENTION

In some aspects, provided herein are methods of treating or preventing anaphylaxis in a subject. In some embodiments, provided herein is a method of treating or preventing anaphylaxis in a subject, comprising providing to the subject a composition comprising an inhibitor of SerpinA3 gene expression. The composition may comprise an inhibitor of SerpinA3f, SerpinA3g, SerpinA3h, and/or Serpina3i gene expression. In some embodiments, the inhibitor of SerpinA3 gene expression comprises a nucleic acid inhibitor. For example, the inhibitor may comprise siRNA, shRNA, miRNA, gRNA, or crDNA. The composition may be provided to the subject prior to exposure to a potential allergen in order to prevent anaphylaxis in the subject. In some embodiments, the allergen is a food allergen.

In other embodiments, provided herein is a method of treating or preventing anaphylaxis in a subject, comprising providing to the subject a composition comprising an inhibitor of a protein encoded by a SerpinA3 gene. The composition may comprise an inhibitor of a protein encoded by SerpinA3f, SerpinA3g, SerpinA3h, and/or Serpina3i. In some embodiments, the inhibitor is an antibody, antibody fragment, aptamer, or a small molecule. In some embodiments, the composition is provided to the subject following exposure to an allergen to treat or prevent anaphylaxis in the subject. In other embodiments, the composition is provided to the subject prior to exposure to an allergen to prevent anaphylaxis in the subject. In some embodiments, the allergen is a food allergen.

### BRIEF DESCRIPTION OF THE DRAWINGS

FIG. 1 shows the experimental regime for IL-4C injection, VE isolation, and gene expression analysis.

FIG. 2 describes the FACS sorting approach for VE cell isolation. Vascular endothelial cells were identified by forward scatter (FSC-A), side scatter (SSC-A) lineage (Lin)-, EP-CAM⁻ CD31^{hi} hematopoietic markers.

FIG. 3A is a heat map showing the significantly dis-regulated 104 genes (82 genes upregulated in 22 downregulated) in vascular endothelial (VE) cells derived from control and IL-4 complex (IL-4+anti-IL-4mAb) treated mice. (N=three mice per group). FIG. 3B shows the RPKM value of the 12 most upregulated genes in VE cells from control and IL-4 complex (IL-4+anti-IL-4mAb) treated mice. Dated described as the mean±SD; n=3 mice per group. Additional results are graphically represented in FIG. 6.

FIG. 4 shows quantitative RT-PCR analysis for VE-cadherin and Von Willebrand factors (VWF) in purified vascular endothelial cells isolated by protocol described in FIG. 2 and nine endothelial epithelial cells and hemopoietic cells. Dated described as the mean±SD; n=3 mice per group.

FIG. 5 shows a heat map demonstrating significant difference in expression of genes as part of the venous endothelial cell, vascular endothelial cell, arterial endothelial cell and hemopoietic cell transcriptome to identify the endothelial cell composition of the purified population described in FIG. 2 and FIG. 3.

FIG. 6 shows RPKM expression level of Serpin A3 family members (Serpin A3f, Serpin A3g, Serpin A3h, Serpin A3i, Serpin A3n) in lung vascular endothelial cells purified from vehicle treated and IL-4 complex treated mice as described in FIG. 2.

FIG. 7 shows gene ontology analysis of upregulated genes (82 genes) from purified vascular endothelial cells from IL-4 treated mice.

FIG. 8 shows qPCR validation results of the trends in gene expression observed by RNA sequencing analysis. Relative gene expression as determined by qPCR is shown in the top row, whereas the RNA sequencing results are shown in the bottom row.

FIG. 9 shows various strategies attempted to generate the SerpinA3 knockout mice.

FIG. 10 shows a schematic exemplifying a suitable gRNA/Cas9-based method for generating SerpinA3 knock-out mouse models.

FIG. 11 shows genomic sequencing data of the Serpin A3 gene locus from Crispr/Cas targeted mice.

FIG. 12A-C show huSerpinA3 expression in primary Human Umbilical Vein Cells: (HUVEC) (FIG. 12A), Immortalized Human Microvascular Endothelial Cells: (HMEC-1)(FIG. 12B), and Immortalized HUVEC: (EAhy 926) (FIG. 12C) following contact with vehicle or huIL-4 (100 ng/ml).

FIG. 13 shows that histamine- and IL-4-induced vascular endothelial barrier dysfunction is SerpinA3-dependent. FIG. 13A shows volume of lentiviral particles used to knockdown SerpinA3 in EAhy926 cells. FIG. 13B is a graph showing HRP flux of EAhy926 (human umbilical vein cell line) cells and EAhy926 cells transduced with SerpinA3 shRNA or empty vector (PLKO) pretreated with IL-4 (100 ng/mL) and stimulated with histamine (100 μM). Data are represented as means±SDs. Symbol represents individual well. *p<0.05, **p<0.01, and ****p<0.0001. ns, p>0.05.

FIG. 14A-14B show IL-4 induction of human Serpin A3 in human endothelial cell lines. FIG. 14A shows Human Serpin A3 mRNA expression in HMEC-1 (human microvascular endothelial cell-1) cells, and FIG. 14B shows expression in HUVEC (human umbilical vein endothelial) cells. For each panel, expression is shown following twenty four hours IL-4 (100 ng/ml). EC cells were stimulated with IL-4 (100 ng/ml) for twenty four hours and cell mRNA expression of SerpinA3 was analyzed by qRT-PCR analyses. Individual symbols represent single well and column (mean) and error bars (standard deviation). *p<0.05.

FIG. 15A-B show that SerpinA3 deletion attenuated hypovolemic shock in a passive IgE-mediated anaphylaxis. FIG. 15A shows maximum temperature change and FIG. 15B shows hematocrit percentage in WT (C57BL/129) and SerpinA3−/− WT mice. Mice were injected intravenously (i.v.) with IL-4C (1 μg of IL-4 plus 5 μg of anti-IL-4 mAb) and i.v. with anti-IgE (EM95; 1 ug/200 ul) twenty four hours later, and anaphylaxis was assessed. Data are represented as means±SD (n=4-13 mice per group). *p<0.05, and ****p<0.0001. ns, p>0.05.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “allergen” refers to any substance that is capable of inducing an allergic reaction in a subject. The term “allergen” as used herein is used in the broadest sense with relation to any allergen, including food allergens (e.g. peanuts, tree nuts, eggs, milk, shellfish, wheat, etc.), environmental allergens (dust, pollen, pet dander, mold, insect bites, etc.), and the like. In some embodiments, the term “allergen” refers to a food allergen, such as peanuts or tree nuts. The term “food allergen” refers to an allergen found in a food or beverage product.

As used herein, the term “anaphylaxis” refers to a broad class of immediate-type hypersensitivity and anaphylactic conditions well known to those skilled in the art including, but not limited to, anaphylactoid reactions, anaphylactic shock, idiopathic anaphylaxis, allergen induced anaphylaxis, exercise induced anaphylaxis, exercise-induced food-dependent anaphylaxis, active anaphylaxis, aggregate anaphylaxis, antiserum anaphylaxis, generalized anaphylaxis, inverse anaphylaxis, local anaphylaxis, passive anaphylaxis, reverse anaphylaxis, and systemic anaphylaxis. In some embodiments, “anaphylaxis” refers to food-induced anaphylaxis, induced by an allergen found in a food or beverage product. An “episode” of anaphylaxis, as that term is used herein, refers to a continuous manifestation of anaphylaxis in a patient.

As used herein, the term “antibody” refers to an immunoglobulin molecule that is typically composed of two identical pairs of polypeptide chains, each pair having one “light” (L) chain and one “heavy” (H) chain. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. Each heavy chain is comprised of a heavy chain variable region (abbreviated herein as HCVR or V_{H}) and a heavy chain constant region. The heavy chain constant region is comprised of three domains, C_{H1}, C_{H2} and C_{H3}. Each light chain is comprised of a light chain variable region (abbreviated herein as LCVR or V_{L}) and a light chain constant region. The light chain constant region is comprised of one domain, CL. The constant regions of the antibodies may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of each heavy/light chain pair (V_{H} and V_{L}), respectively, form the antibody binding site. The term “antibody” encompasses an antibody that is part of an antibody multimer (a multimeric form of antibodies), such as dimers, trimers, or higher-order multimers of monomeric antibodies. It also encompasses an antibody that is linked or attached to, or otherwise physically or functionally associated with, a non-antibody moiety. Further, the term “antibody” is not limited by any particular method of producing the antibody. For example, it includes, inter alia, recombinant antibodies, synthetic antibodies, monoclonal antibodies, polyclonal antibodies, bi-specific antibodies, and multi-specific antibodies.

As used herein, the term “antibody derivative” or “derivative” of an antibody refers to a molecule that is capable of binding to the same antigen that the antibody from which it is derived binds to and comprises an amino acid sequence that is the same or similar to the antibody linked to an additional molecular entity. The amino acid sequence of the antibody that is contained in the antibody derivative may be the full-length antibody, or may be any portion or portions of a full-length antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional entity by chemical coupling, genetic fusion, noncovalent association or otherwise.

The term “antibody derivative” also encompasses chimeric antibodies, humanized antibodies, and molecules that are derived from modifications of the amino acid sequences of an antibody, such as conservation amino acid substitutions, additions, and insertions. As used herein, the term “derivative” also refers to protein constructs being structurally different from, but still having some structural relationship to the common antibody concept, e.g., scFv, Fab and/or F(ab)2, as well as bi-, tri- or higher specific antibody constructs or monovalent antibodies, and further retaining target binding capacities.

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

As used herein, the term “antigen-binding fragment” of an antibody refers to one or more portions of a full-length antibody that retain the ability to bind to the same antigen that the antibody binds to.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

The terms “buffer” or “buffering agents” refer to materials, that when added to a solution, cause the solution to resist changes in pH.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “Fab” relates to an IgG fragment comprising the antigen binding region, said fragment being composed of one constant and one variable domain from each heavy and light chain of the antibody.

As used herein, the term “F(ab)2” relates to an IgG fragment consisting of two Fab fragments connected to one another by one or more disulfide bonds.

As used herein, the term “inhibit”, “inhibition”, and variations thereof refer to reducing or completely eliminating the activity or expression of an entity (e.g. a gene, a protein, etc.). In some embodiments, inhibition of gene expression refers to silencing of a gene. In some embodiments, inhibition of gene expression refers to a reduction in gene expression. In some embodiments, inhibition of a protein refers to a reduction or complete elimination of the activity of that protein.

As used herein, the term “isolated antibody” or “isolated binding molecule” refers to an antibody or binding molecule that is identified and separated from at least one contaminant with which it is ordinarily associated in its source. Examples of an isolated antibody include: an antibody that: (1) is not associated with one or more naturally associated components that accompany it in its natural state; (2) is substantially free of other proteins from its origin source; or (3) is expressed recombinantly, in vitro, or cell-free, or is produced synthetically and the is removed the environment in which it was produced.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

The terms “pharmaceutically acceptable” or “pharmacologically acceptable” as used herein, refer to compositions that do not substantially produce adverse reactions (e.g., toxic, allergic or immunological reactions) when administered to a subject.

As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethyl glycol, other natural and non-naturally occurring carries, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the terms “prevent,” “prevention,” and preventing” refer to reducing the likelihood of a particular condition or disease state (e.g., anaphylaxis) from occurring in a subject not presently experiencing or afflicted with the condition or disease state. The terms do not necessarily indicate complete or absolute prevention. For example “preventing anaphylaxis” refers to reducing the likelihood of anaphylaxis occurring in a subject not presently experiencing or anaphylaxis. In order to “prevent anaphylaxis” a composition or method need only reduce the likelihood of anaphylaxis, not completely block any possibility thereof “Prevention,” encompasses any administration or application of a therapeutic or technique to reduce the likelihood of anaphylaxis developing (e.g., in a mammal, including a human). Such a likelihood may be assessed for a population or for an individual.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide.

As used herein, the terms “treat,” “treatment,” and “treating” refer to reducing the amount or severity of a particular condition, disease state (e.g., anaphylaxis), or symptoms thereof, in a subject presently experiencing or afflicted with the condition or disease state. The terms do not necessarily indicate complete treatment (e.g., total elimination of the anaphylaxis). “Treatment,” encompasses any administration or application of a therapeutic or technique for a condition (e.g., in a mammal, including a human), and includes inhibiting the condition, arresting its development, relieving the condition, causing regression, or restoring or repairing a lost, missing, or defective function; or stimulating an inefficient process.

As used herein, the term “sample” is used in its broadest sense and encompass materials obtained from any source. As used herein, the term “sample” is used to refer to materials obtained from a biological source, for example, obtained from animals (including humans), and encompasses any fluids, solids and tissues.

As used herein, the term “subject” refers to any human or animal (e.g., non-human primate, rodent, feline, canine, bovine, porcine, equine, etc.). In some embodiments, the subject is a human.

### DETAILED DESCRIPTION OF THE INVENTION

The present disclosure relates to novel therapeutic targets for the treatment and/or prevention of anaphylaxis in a subject. In particular, the present disclosure identifies inhibition of Serpin family A member 3 (SerpinA3) as a novel therapeutic target for the treatment and/or prevention of anaphylaxis in a subject.

The protein encoded by the SerpinA3 gene is a plasma protease inhibitor and member of the serine protease inhibitor class. The results presented herein demonstrate a role for SerpinA3 family members in the treatment or prevention of anaphylaxis. Accordingly, in some embodiments provided herein is a method treating or preventing anaphylaxis in a subject. In some embodiments, provided herein is a method of treating or preventing food-induced anaphylaxis in a subject. The food-induced anaphylaxis may be induced by any food allergen. Examples of common food allergens that may induce anaphylaxis include peanuts, tree nuts, fish, shellfish, milk, eggs, soy, and wheat.

In some embodiments, the method of treating or preventing anaphylaxis in a subject comprises providing to the subject a therapeutic agent. In some embodiments, the therapeutic agent is an inhibitor of SerpinA3 gene expression or an inhibitor of a protein encoded by a SerpinA3 gene. The inhibitor may reduce or silence the expression of any SerpinA3 gene (e.g. SerpinA3f, SerpinA3G, SerpinA3h, SerpinA3i, etc.) or may reduce or completely eliminate the activity of a protein encoded by a SerpinA3 gene (e.g. a protein encoded by SerpinA3f, SerpinA3G, SerpinA3h, SerpinA3i, etc.). For example, the inhibitor may reduce or eliminate the binding of a protein encoded by a SerpinA3 gene to a serine protease.

For general success, suitable therapeutic agents should possess minimal general cell toxicity. Furthermore, agents should be cell permeable and/or amenable to cell-specific delivery. To facilitate cell permeability, suitable delivery vectors may be used. For example, delivery systems, such as vectors (e.g. viral or bacterial vectors), or non-viral vectors (e.g. plasmids, liposomes, nanoparticles, etc.) may be used.

Suitable methods of inhibiting or reducing SerpinA3 gene expression include, for example, nucleic acid inhibitors. For example, RNA interference technologies may be used to reduce or inhibit SerpinA3 gene expression. For example, RNA interference (RNAi) technologies including siRNA, shRNA, or miRNA may be used to reduce or silence expression of a SerpinA3 gene. Suitable sequences for nucleic acid inhibitors may be designed based upon the sequence of the SerpinA3 gene.

siRNA are short artificial RNA molecules which can be chemically modified to enhance stability. Because siRNA are double-stranded, the principle of the ‘sense’ and the ‘antisense’ strand also applies. The sense strands have a base sequence identical to that of the transcribed mRNA and the antisense strand has the complementary sequence. An siRNA molecule administered to a patient is bound by an intracellular enzyme called Argonaut to form a so-called RNA-induced silencing complex (RISC). The antisense strand of the siRNA guides RISC to the target mRNA, where the antisense strand hybridizes with the target mRNA, which is then cleaved by RISC. In such way, translation of the respective mRNA is interrupted. The RISC can then cleave further mRNAs. Suitable delivery technologies for siRNA include chemical modifications, lipid-based nanovectors, polymer-mediated delivery systems, conjugate delivery systems, and the like.

shRNA is an artificial RNA molecule with a tight hairpin turn that can be used to silence target gene expression via RNA interference (RNAi). shRNA can be delivered to cells, e.g., by means of a plasmid or through viral or bacterial vectors. shRNA is an advantageous mediator of RNAi in that it has a relatively low rate of degradation and turnover. Plasmids for shRNA delivery may comprise a suitable promoter to express the shRNA. Any suitable promoter may be used, such as a polymerase promoter. Once the plasmid or vector has integrated into the host genome, the shRNA is transcribed in the nucleus. The product mimics pri-microRNA (pri-miRNA) and is processed by Drosha. The resulting pre-shRNA is exported from the nucleus by Exportin 5. This product is then processed by Dicer and loaded into the RNA-induced silencing complex (RISC), after which the same silencing follows as in siRNA.

microRNA (abbreviated miRNA) is a small non-coding RNA molecule (containing about 22 nucleotides) found in plants, animals and some viruses, that functions in RNA silencing and post-transcriptional regulation of gene expression.

In some embodiments, a CRISPR system may be used to inhibit SerpinA3 gene expression. Suitable CRISPR systems include CRISPR/Cas systems, CRISPR/Cpf1 systems, and the like. For example, a nucleic acid based inhibitor may be the guide RNA of a CRISPR system (e.g. a CRISPR/Cas system, a CRISPR/Cpf1 system, etc.). In some embodiments, guide RNA (gRNA) comprises a target-specific crRNA (“small interfering CRISPR RNA”) capable of hybridizing with a genomic strand of the SerpinA3 gene. Alternatively, a nucleic acid based inhibitor can be the crRNA alone. The guide RNA crRNA is capable of directing the endonuclease (e.g. Cas enzyme, Cpf1 enzyme, or other endonuclease), to the SerpinA3 gene, where the endonuclease carries out sequence specific strand breaks. By creating one or more double strand breaks, the SerpinA3 gene hence can be silenced.

In some embodiments, the inhibitor of SerpinA3 gene expression may be interfere with translation of messenger RNA into protein (e.g., antisense oligonucleotides, peptide nucleic acids (PNAs), ribozymes, deoxyribozymes, etc.) In some embodiments, the inhibitor may directly interfere with gene transcription (e.g., triple helix-forming oligonucleotides, peptide nucleic acids, decoy molecules (linear or circular), synthetic minor groove-binding ligands, etc.).

Other suitable methods for inhibiting or reducing SerpinA3 gene expression include DNA or RNA binding agents. For example, suitable DNA binding agents include minor groove-binding ligands, intercalating ligands (e.g. metallointercalators), and polyamides (e.g. pyrrole-imidazole polyamides).

In some embodiments, the method comprises providing to the subject an inhibitor of the protein encoded by a SerpinA3 gene. Suitable inhibitors may inhibit the activity of the protein. For example, suitable inhibitors for reducing protein activity include antibodies, antibody fragments, aptamers, and small molecules.

Aptamers are oligonucleotides that have specific binding properties for a pre-determined target. They may be obtained from a randomly synthesized library containing up to 10¹⁵ different sequences through a combinatorial process named SELEX (“Systematic Evolution of Ligands by Exponential enrichment”). Aptamer properties are dictated by their 3D shape, resulting from intramolecular folding, driven by their primary sequence. An aptamer3D structure is exquisitely adapted to the recognition of its cognate target through hydrogen bonding, electrostatic and stacking interactions. Aptamers generally display high affinity (Kd about micromolar (μM) for small molecules and picomolar (pM) for proteins). An overview on the technical repertoire to generate target specific aptamers is described in Mol Ther Nucleic Acids. 2015 January; 4(1), the entire contents of which are incorporated herein by reference.

In some embodiments, the antagonist or inhibitor is a small molecule. Preferably, said small molecule is an organic molecule, and/or said small molecule has a molecular weight of smaller <550 DA, preferably <500 DA, more preferably <450 DA.

The therapeutic agent may formulated into a composition, optionally comprising one or more pharmaceutically acceptable carriers. Such a composition may be provided to the subject by any suitable route. In some embodiments, compositions are formulated for administration by any suitable route, including but not limited to, orally (e.g., such as in the form of tablets, capsules, granules or powders), sublingually, bucally, parenterally (such as by subcutaneous, intravenous, intramuscular, intradermal, or intracisternal injection or infusion (e.g., as sterile injectable aqueous or non-aqueous solutions or suspensions, etc.)), nasally (including administration to the nasal membranes, such as by inhalation spray), topically (such as in the form of a cream or ointment), transdermally (such as by transdermal patch), rectally (such as in the form of suppositories), etc.

The therapeutic agent may be provided to the subject at any suitable time point to treat or prevent anaphylaxis in the subject. For example, the therapeutic agent may be provided to the subject following exposure to an allergen which may cause anaphylaxis in the subject. For example, the therapeutic agent may be provided to the subject within 30 seconds, within 1 minute, within 2 minutes, within 3 minutes, within 4 minutes, within 5 minutes, within 10 minutes, within 15 minutes, within 20 minutes, within 25 minutes, within 30 minutes, within 45 minutes, or within 1 hour of exposure to the allergen. Such embodiments may be particularly useful when the therapeutic agent is an inhibitor of activity of a protein encoded by a SerpinA3 gene (e.g. an antibody, antibody fragment, aptamer, small molecule, etc.).

In some embodiments, the therapeutic agent may be provided to the subject prior to exposure to an allergen which may cause anaphylaxis in the subject. For example, the therapeutic agent may be provided to the subject prior to exposure to the allergen to prevent anaphylaxis in the subject. Such embodiments may be particularly useful when the therapeutic agent is an inhibitor of SerpinA3 gene expression (e.g. shRNA, siRNA, miRNA, CRISPR-based technologies, etc.). For example, an inhibitor of SerpinA3 gene expression may be provided to the subject to reduce or silence expression of the SerpinA3 gene, thereby preventing (e.g. reducing the risk of) the subject developing anaphylaxis following exposure to an allergen. For example, cells may be isolated from a subject, gene therapy (e.g. RNAi based technologies) may be performed on cells isolated and cultured ex vivo, those cells may be re-introduced back into the subject to prevent future episodes of anaphylaxis in the subject. Such embodiments may also be useful when the therapeutic agent is an inhibitor of a protein encoded by a SerpinA3 gene (e.g. an antibody, antibody fragment, aptamer, small molecule, etc.), which may similarly be provided to the subject prior to exposure to an allergen to prevent anaphylaxis in the subject. For example, an inhibitor that reduces function of a protein encoded by a SerpinA3 gene (e.g. reduces or eliminates binding of a serine protease inhibitor to serine proteases) may be provided to the subject prior to exposure to an allergen to prevent anaphylaxis in the subject.

The therapeutic agent may be provided to the subject once or multiple times. In some embodiments, the therapeutic agent may be provided to the subject over the course of multiple, scheduled intervals to prevent anaphylaxis in the subject. The therapeutic agent may co-administered with other suitable therapies to treat and/or prevent anaphylaxis in the subject. For example, the therapeutic agent may be used in combination with oral immunotherapy to prevent anaphylaxis in a subject. Oral immunotherapy (OIT) refers to feeding an allergic individual an increasing amount of an allergen with the goal of increasing the threshold that triggers a reaction. For example, a person allergic to peanuts may be given very small amounts of peanut protein that would not trigger a reaction. This small amount is gradually increased (e.g. in the allergist's office or a clinical research setting) over a period of time (e.g. months). The goal of therapy is to raise the threshold that may trigger a reaction and provide the allergic individual protection against accidental ingestion of the allergen.

The mode of administration, frequency of administration, timing of administration, and dose of the therapeutic agent depend on the type of therapeutic agent (e.g. antibody, aptamer, small molecule, nucleic acid inhibitor, etc.), the age of the subject, the weight of the subject, the intended result of administration (e.g. treatment or prevention of anaphylaxis), and the like.

Methods for determining the efficacy of a suitable therapeutic agent (e.g. the ability of the agent to inhibit SerpinA3 gene expression or to reduce activity of a protein encoded by a SerpinA3 gene) are known in the art. For example, SerpinA3 gene expression may be determined by measuring/quantifying SerpinA3 nucleic acid (e.g. RNA sequencing, PCR methods, etc.). As another example, SerpinA3 gene expression may be inferred based upon levels of protein encoded by a SerpinA3 gene, which may be quantified by a suitable assay (e.g. western blot, ELISA, mass spectrometry, etc.). As another example, activity of a protein encoded by a SerpinA3 gene may be quantified, such as by an HRP assay to determine endothelial barrier function. Suitable methods for determining the efficacy of a therapeutic agent are described in Example 1 below.

### EXPERIMENTAL

The following examples are provided in order to demonstrate and further illustrate certain preferred embodiments and aspects of the present invention and are not to be construed as limiting the scope thereof.

### Example 1

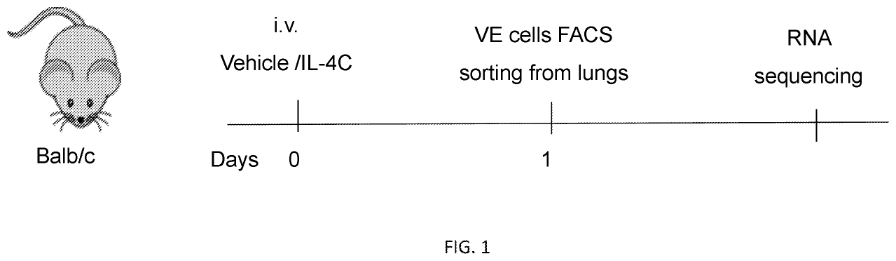

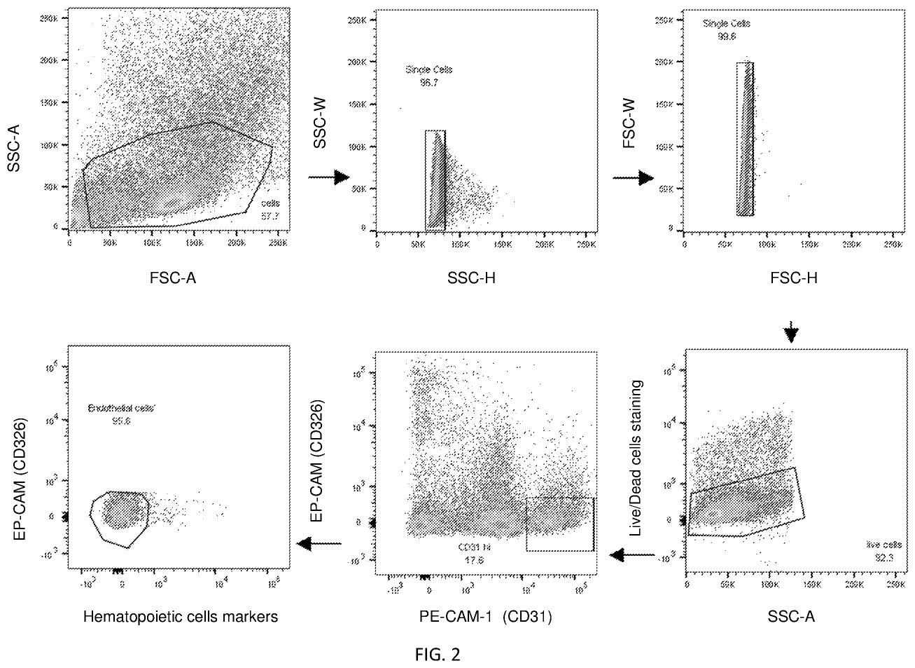

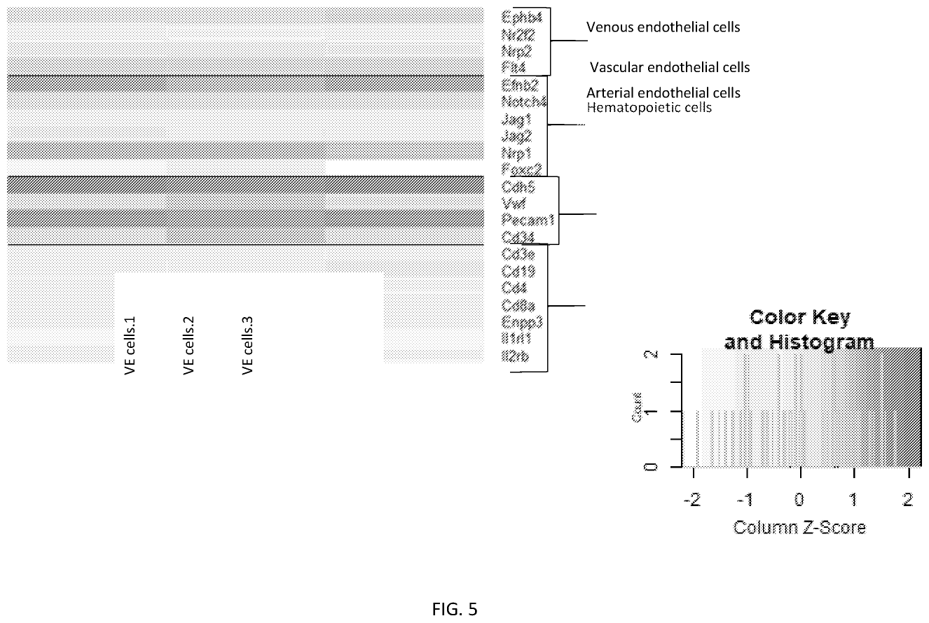

The experiments conducted herein were designed to elucidate the underlying cellular and molecular pathways regulated by IL-4 to enhance vascular endothelial dysfunction associated with food-induced anaphylaxis. WT BALB/c mice were used for VE cell isolation procedures. The timeline for VE isolation is shown in FIG. 1. Mice were injected intravenously with vehicle or with IL-4C (recombinant, IL-4-neutralizing, anti-IL-4 monoclonal antibody [mAb] complex, 1:5 weight [1 μg of IL-4+5 μg anti-IL-4 mAb]). Twenty-four hours later, mice were sacrificed and the lungs were harvested. Cells were lysed, and VE cells were obtained by FACS sorting using EP-CAM⁻ CD31^{hi} hematopoietic markers (FIG. 2). As shown in FIG. 2 and FIG. 5, cells show a distinct transcriptome profiles for VE cells.

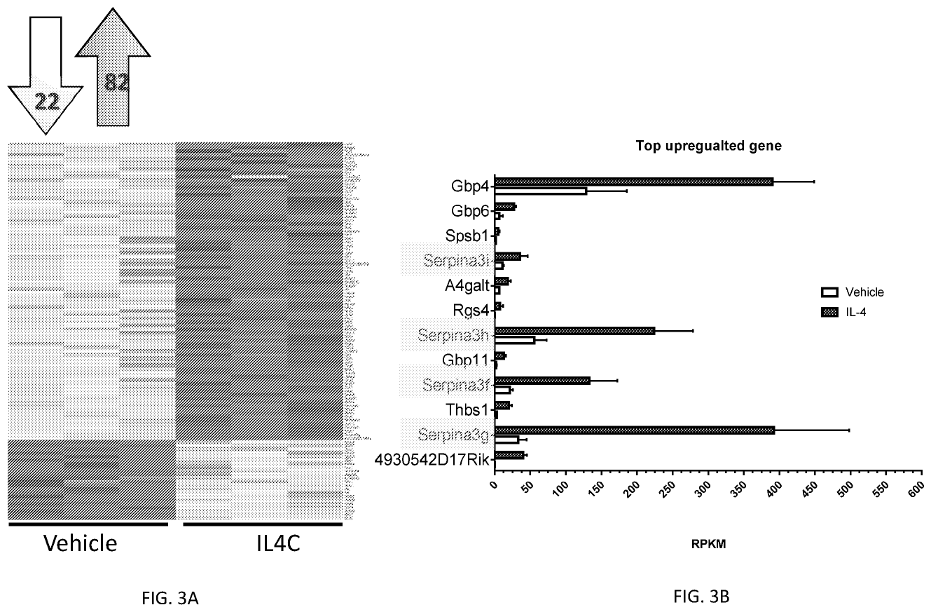

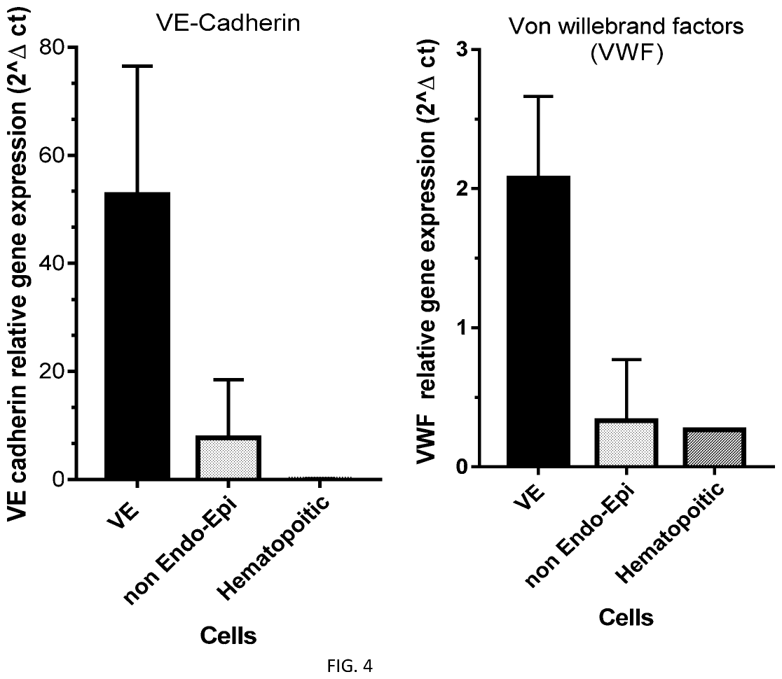

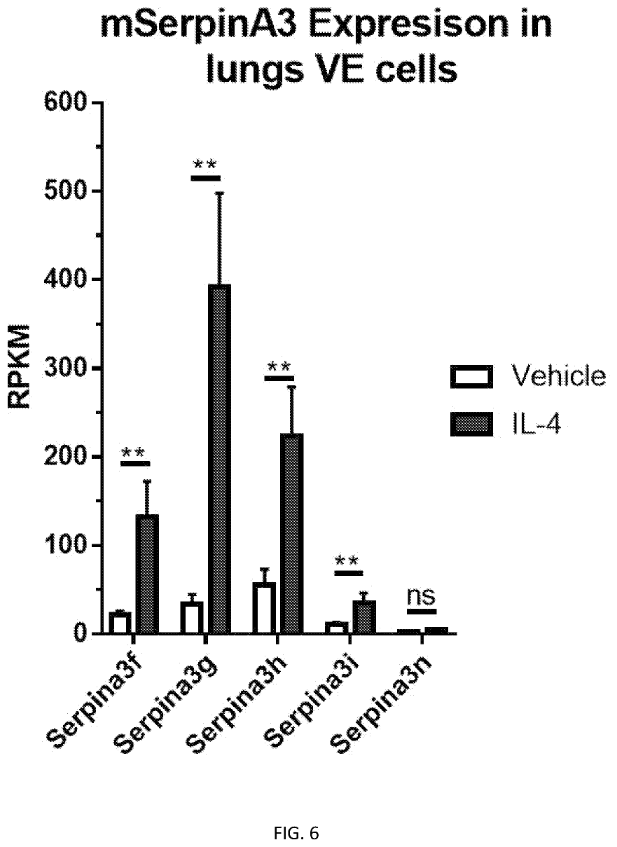

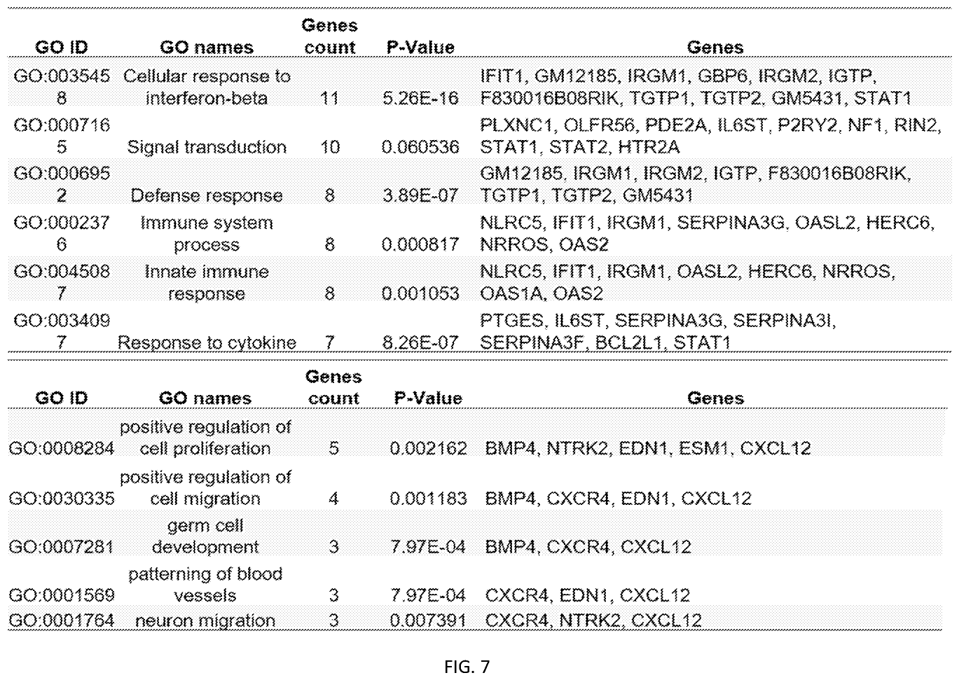

RNA sequencing was performed to identity relative gene expression of various processes regulated by IL4 in the VE cells. Levels of VE-cadherin and Von Willebrand factors (VWF) are shown in FIG. 4. Levels of other top upregulated genes in VE cells from IL-4C injected mice are shown in FIG. 3. Results are graphically represented in the panel on the right. As shown in FIG. 3, right panel, SerpinA3i, SerpinA3h, SerpinA3f, and SerpinA3g are significantly upregulated in mice injected with IL-4C compared to vehicle. Additional results are graphically represented in FIG. 6.

Gene ontology analysis of IL-4 dysregulated genes is shown in FIG. 7.

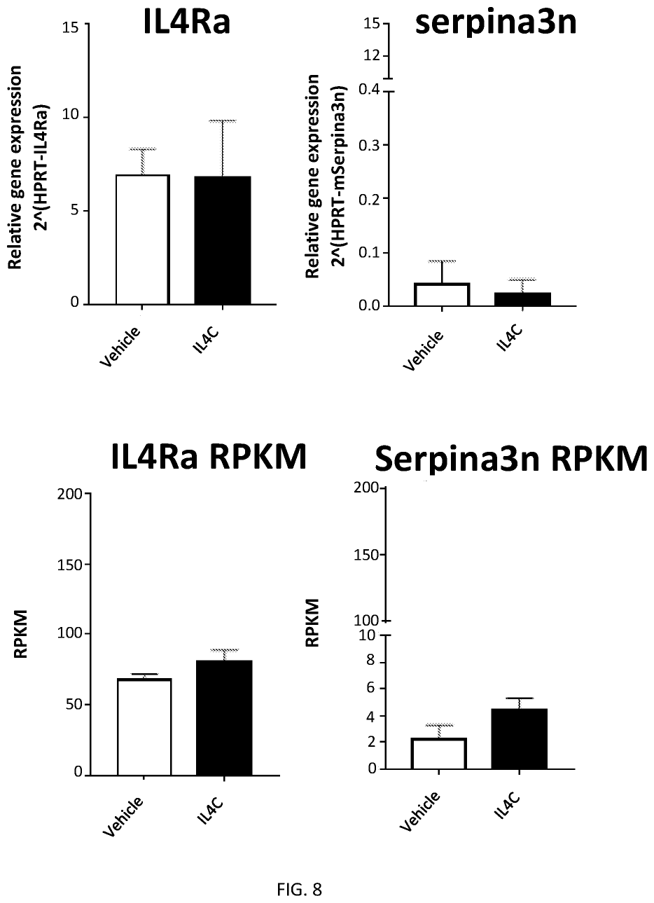

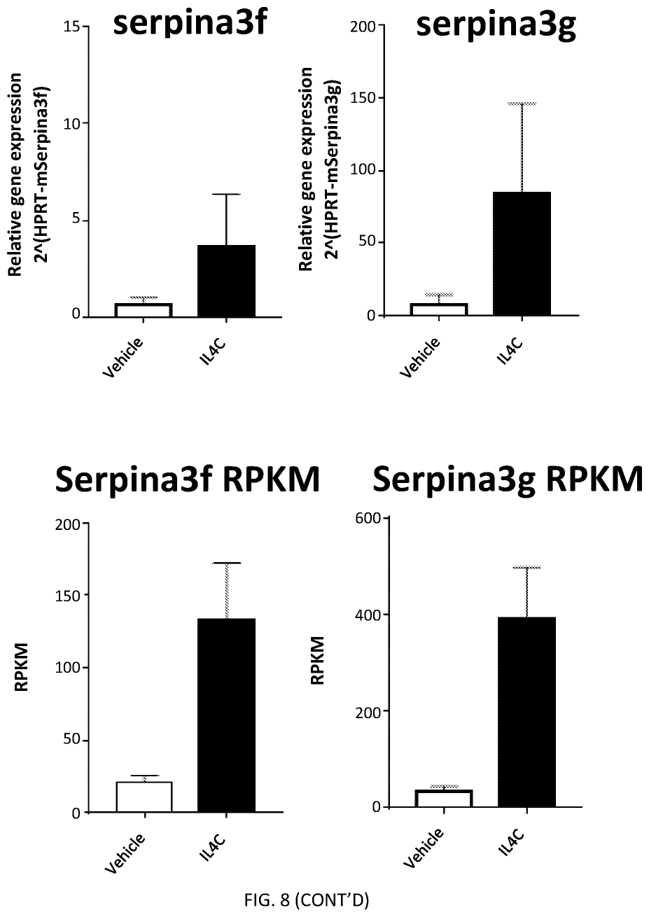

To confirm the trends in gene expression observed by RNA sequencing analysis, qPCR was performed to assess gene expression of Il4a, SerpinA3n, SerpinA3f, and SerpinA3g. Validation results are FIG. 8. Relative gene expression as determined by qPCR is shown in the top row, whereas the RNA sequencing results are shown in the bottom row. As shown in the figure, levels of SerpinA3f and SerpinA3g were shown to be significantly enhanced in both RNA sequencing and qPCR analysis.

IL-4 and histamine-induced vascular endothelial cell barrier dysfunction and fluid extravasation and the onset of a severe food-induced anaphylactic reaction has been shown to be dependent on a VE STAT3. Moreover, STAT3 has been shown to bind to regulatory elements in the promoters of SerpinA3. Accordingly, SerpinA3 was further investigated as a potential regulator of the anaphylactic response.

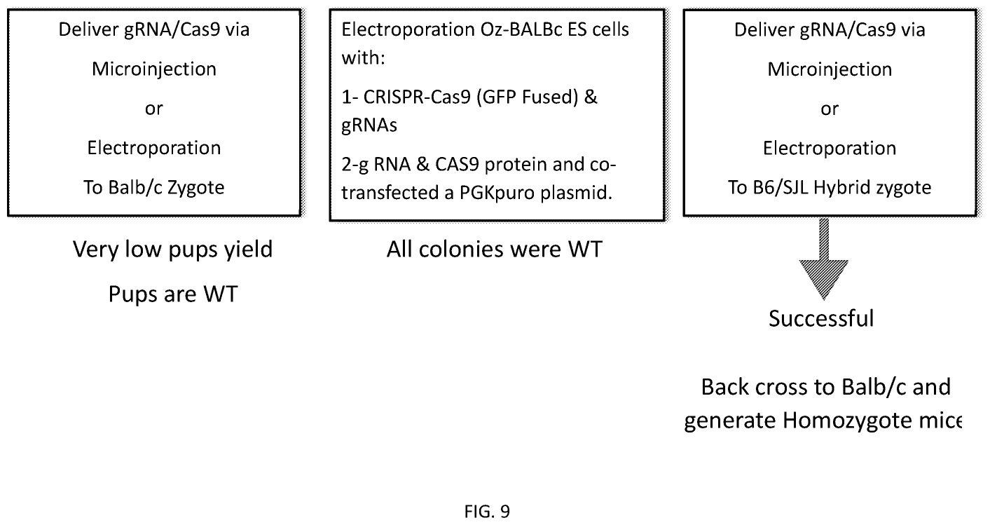

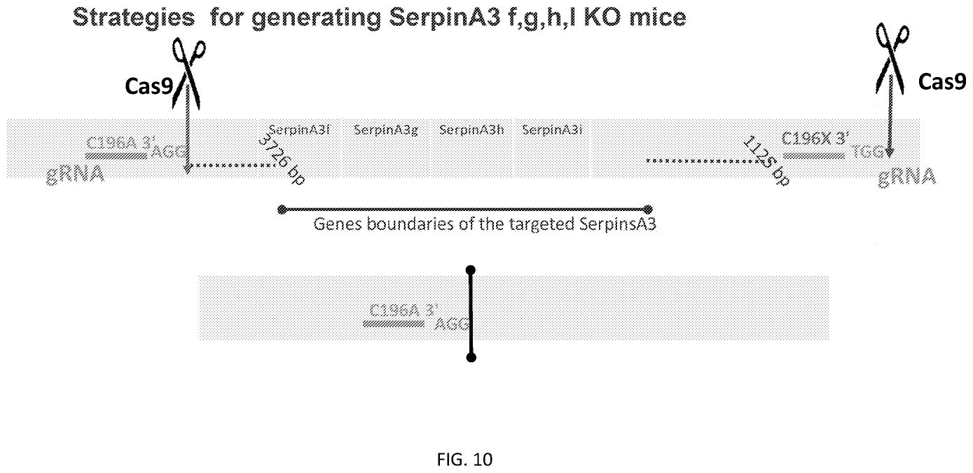

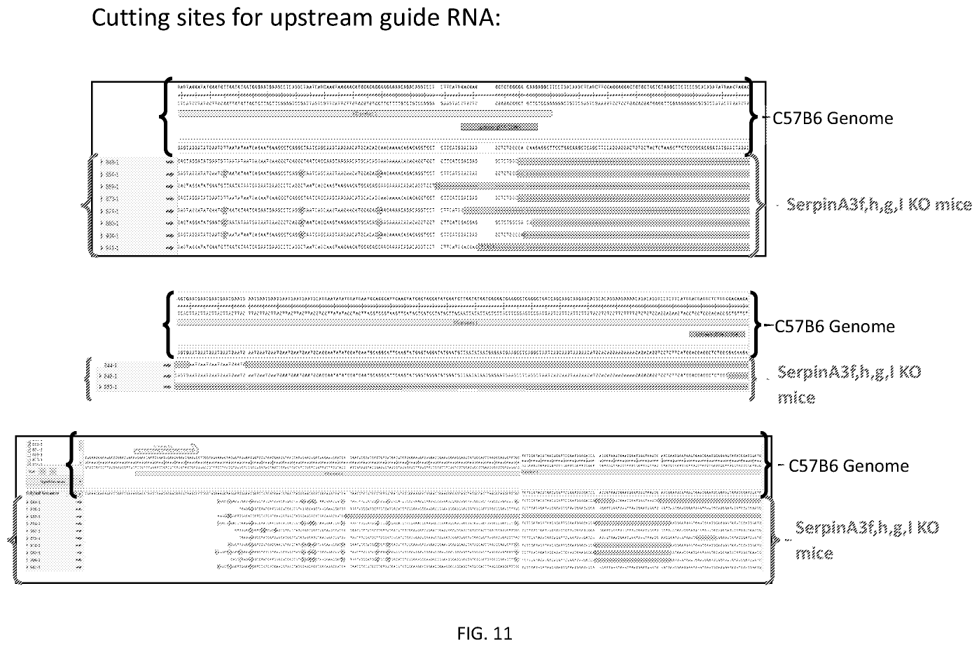

Additional experiments were conducted to define the requirement of vascular endothelial SerpinA3 in IL-4 and histamine-induced anaphylaxis and vascular leakage, examine the requirement of VE huSerpinA3 in IL-4 and histamine-induced endothelial barrier dysfunction, and determine the role of STAT3-SerpinA3 axis in IL-4 and histamine-induced vascular leakage. To accomplish this, Serpina3f, Serpina3g, Serpina3h, and Serpina3l knockout mice were generated. Various strategies were attempted to generate the knockout mice (FIG. 9). Delivery of gRNA/Cas9 via microinjection or electroporation was found to be a successful method and was used for subsequent experiments. Additional details regarding gRNA/Cas9 methods are shown in FIG. 10. Exemplary cutting sites for upstream gRNAs are shown in FIG. 11.

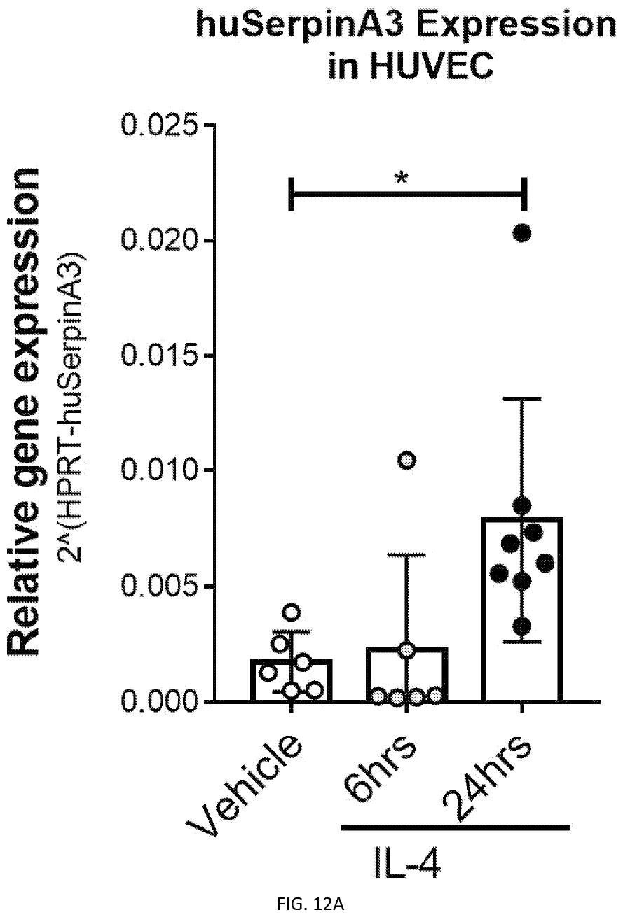

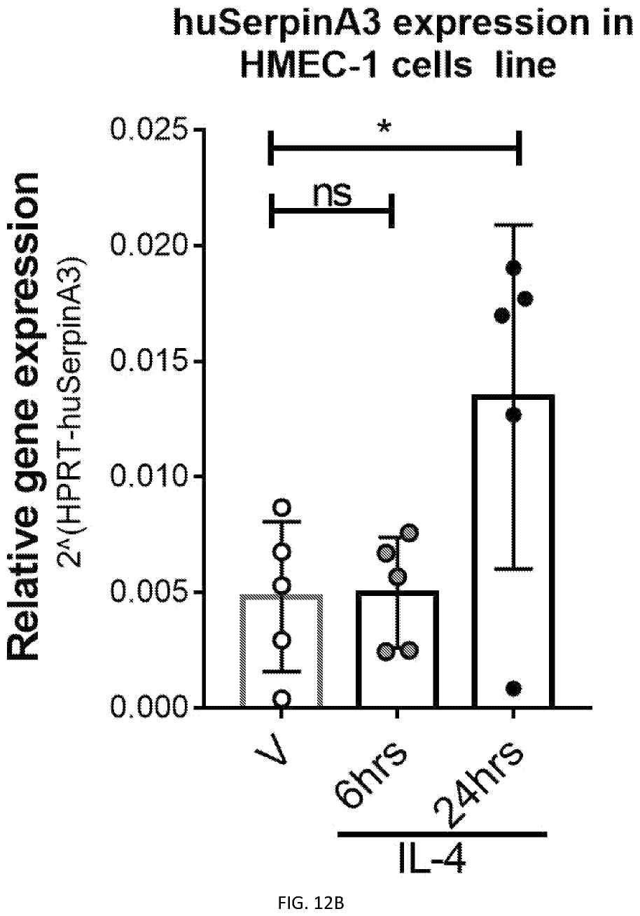

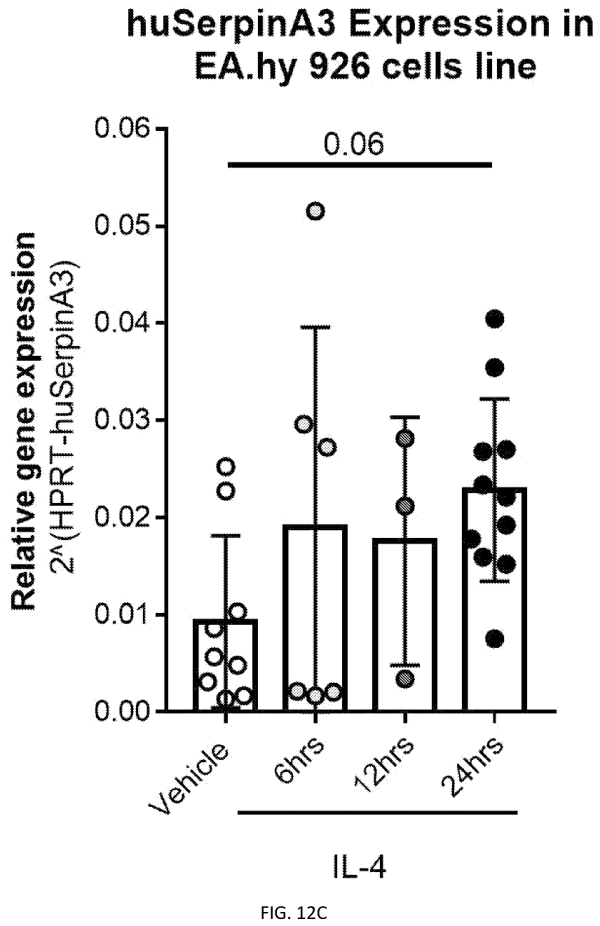

To examine the requirement of VE huSerpinA3 in IL-4 and Histamine-induced endothelial barrier dysfunction, Primary Human Umbilical Vein Cells: (HUVEC), Immortalized HUVEC: (EAhy 926), and Immortalized Human Microvascular Endothelial Cells: (HMEC-1) were used. Cells were contacted with vehicle or huIL-4 (100 ng/ml) and huSerpinA3 expression was evaluated after various time periods. For example, huSerpinA3 expression was measured by PCR in the experimental results shown in FIG. 12A-C. Taken together, these results show that IL4 Treatment enhances the expression of VE SerpinA3 (g,f,h,i) genes in mice (Balb/c) and SerpinA3human cell lines (HUVEC-HMEC-1).

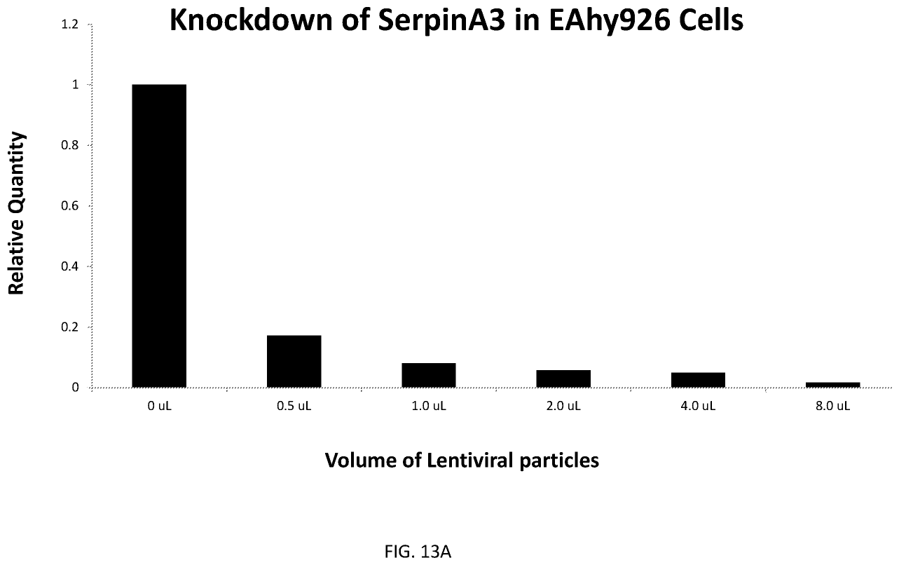

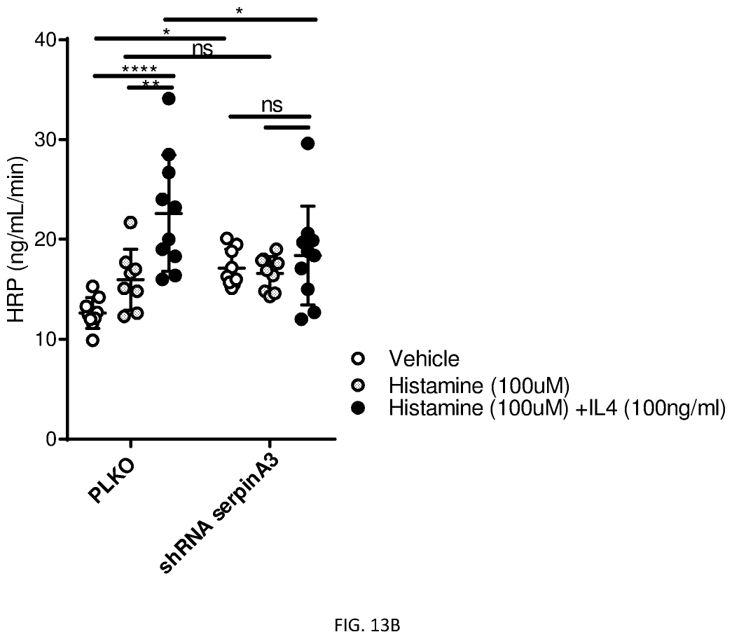

Alternatively or in addition, vascular endothelial cells may be contacted with vehicle, histamine, or histamine and IL-4, and the endothelial barrier function may be assessed by a suitable assay, such as an HRP assay. For example, such an experiment is shown in FIG. 13, demonstrating HRP analysis in EAhy926 cell lines following contact with a vehicle, histamine, and histamine+IL-4. FIG. 13B shows that histamine- and IL-4-induced vascular endothelial barrier dysfunction is SerpinA3-dependent. The graph shows HRP flux of EAhy926 (human umbilical vein cell line) cells and EAhy926 cells transduced with SerpinA3 shRNA or empty vector (PLKO) pretreated with IL-4 (100 ng/mL) and stimulated with histamine (100 μm).

To determine the role of STAT3-SerpinA3 axis in IL-4 and histamine-induced vascular leakage, Immortalized Human Microvascular Endothelial Cells (HMEC-1) and control-vector cells (e.g. STAT3shRNA cells) may be contacted with vehicle or huIL-4 (100 ng/ml). huSerpinA3 expression may be determined, such as by PCR. STAT3 binding genes may be determined by chromatin immunoprecipitation (ChIP) sequencing. It would be expected that IL-4 signaling through the VE STAT3 pathway would increase expression of SerpinA3, leading to enhancement of histamine induced vascular leakages and anaphylaxis.

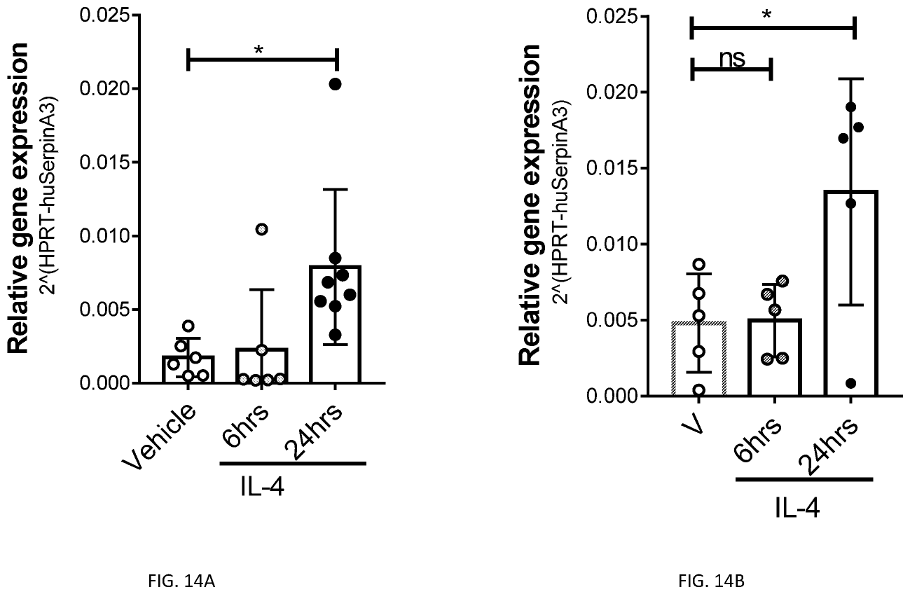

FIG. 14A-14B show IL-4 induction of human Serpin A3 in human endothelial cell lines. FIG. 14A shows Human Serpin A3 mRNA expression in HMEC-1 (human microvascular endothelial cell-1) cells, and FIG. 14B shows expression in HUVEC (human umbilical vein endothelial) cells. For each panel, expression is shown following twenty four hours IL-4 (100 ng/ml). EC cells were stimulated with IL-4 (100 ng/ml) for twenty four hours and cell mRNA expression of SerpinA3 was analyzed by qRT-PCR analyses. Individual symbols represent single well and column (mean) and error bars (standard deviation). *p<0.05.

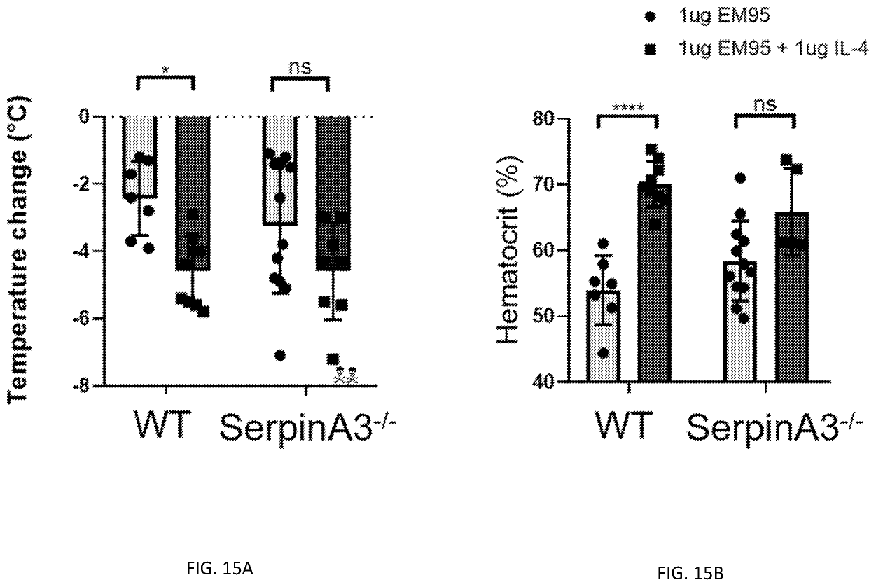

To define the requirement of vascular endothelial SerpinA3 in IL-4 and Histamine-induced anaphylaxis and vascular leakage, the knockout mice generated as described above were injected intravenously with IL-4C, and 24 hours later injected intravenously with histamine to induce an anaphylactic response. Anaphylaxis was evaluated by measuring changes in body temperature (hypothermia) and hematocrit levels (hypovolemic shock). Results are shown in FIG. 15A-B, which show that SerpinA3 deletion attenuated hypovolemic shock in a passive IgE-mediated anaphylaxis. FIG. 15A shows maximum temperature change and FIG. 15B shows hematocrit percentage in WT (C57BL/129) and SerpinA3−/− WT mice. Mice were injected intravenously (i.v.) with IL-4C (1 μg of IL-4 plus 5 μg of anti-IL-4 mAb) and i.v. with anti-IgE (EM95; 1 ug/200 ul) twenty four hours later, and anaphylaxis was assessed.

Taken together, the results described herein demonstrate that IL-4 enhances the severity of histamine-induced hypovolemic shock through IL-4Ra chain signaling on VE cells, IL-4-STAT3 signaling is required for priming of VE cells and hypovolemic shock during severe histamine-mediated reactions, IL-4-STAT3 signaling is required for priming of VE cells and hypovolemic shock during severe IgE-MC-mediated reactions, and IL-4C in vivo treatment dysregulated VE genes associated with STAT3 activity.

Various modification, recombination, and variation of the described features and embodiments will be apparent to those skilled in the art without departing from the scope and spirit of the invention. Although specific embodiments have been described, it should be understood that the invention as claimed should not be unduly limited to such specific embodiments. Indeed, various modifications of the described modes and embodiments that are obvious to those skilled in the relevant fields are intended to be within the scope of the following claims. All publications and patents mentioned in the present application and/or listed below are herein incorporated by reference in their entireties.

1. A method of treating or preventing anaphylaxis in a subject, comprising providing to the subject a composition comprising an inhibitor of SerpinA3 gene expression.

2. The method of claim 1, wherein the composition comprises an inhibitor of SerpinA3f, SerpinA3g, SerpinA3h, and/or Serpina3i gene expression.

3. The method of claim 1 or 2, wherein the inhibitor of SerpinA3 gene expression comprises a nucleic acid inhibitor.

4. The method of claim 3, wherein the inhibitor comprises siRNA, shRNA, miRNA, gRNA, or crDNA.

5. The method of any of the preceding claims, wherein the composition is provided to the subject prior to exposure to a potential allergen in order to prevent anaphylaxis in the subject.

6. The method of claim 5, wherein the allergen is a food allergen.

7. A method of treating or preventing anaphylaxis in a subject, comprising providing to the subject a composition comprising an inhibitor of a protein encoded by a SerpinA3 gene.

8. The method of claim 7, wherein the composition comprises an inhibitor of a protein encoded by SerpinA3f, SerpinA3g, SerpinA3h, and/or Serpina3i.

9. The method of claim 7 or 8, wherein the inhibitor is an antibody, antibody fragment, aptamer, or a small molecule.

10. The method of any one of claims 7-9, wherein the composition is provided to the subject following exposure to an allergen to treat or prevent anaphylaxis in the subject.

11. The method of any one of claims 7-9, wherein the composition is provided to the subject prior to exposure to an allergen to prevent anaphylaxis in the subject.

12. The method of claim 10 or 11, wherein the allergen is a food allergen.

13. The method of any of the preceding claims, wherein the subject is a human.
